(19) United States
(12) Patent Application Publication
(10) Pub. No.: US 2013/0309709 A1
(43) Pub. Date: Nov. 21, 2013

(54) PHOTOUNCAGING-ASSISTED EVALUATION OF LARGE-SCALE SYNAPTIC REORGANIZATION OF BRAIN CIRCUITS

(71) Applicant: The Florida State University Research Foundation, Inc., (US)

(72) Inventor: Sanjay S. Kumar, Tallahassee, FL (US)

(73) Assignee: The Florida State University Research Foundation, Inc., Tallahassee, FL (US)

(21) Appl. No.: 13/875,589

(22) Filed: May 2, 2013

Related U.S. Application Data
(60) Provisional application No. 61/641,433, filed on May 2, 2012.

(51) Int. Cl. G01N 33/50

(52) U.S. Cl. CPC G01N 33/5091
USPC 435/29

(57) ABSTRACT

Photouncaging-assisted evaluation of large-scale synaptic reorganization of brain circuits and methods in use thereof. The current invention utilizes laser-scanning photostimulation in large-scale and with higher accuracy to detect synaptic reorganization in neurological disorders, such as Alzheimer's disease, Parkinson's disease and epilepsy. Using the invention's methodology, disease and non-disease conditions can be determined, and treatments can be personalized and administered more efficiently.

### CROSS-REFERENCE TO RELATED APPLICATIONS

This nonprovisional application is a continuation of and claims priority to provisional application No. 61/641,433, entitled “Photouncaging-Assisted Evaluation of Large-Scale Synaptic Reorganization of Brain Circuits”, filed May 2, 2012 by the same inventor.

### BACKGROUND OF THE INVENTION

1. Field of the Invention

This invention relates, generally, to assessment of neurological conditions in disease and non-disease states. More particularly, it relates to large-scale synaptic evaluations of synaptic reorganization in brains in disease and non-disease states.

2. Description of the Prior Art

Many prevalent neurological disorders, such as Alzheimer's disease, Parkinson's disease and epilepsy, entail significant reorganization of underlying brain circuitry. While techniques such as stereology have enabled quick assessments of neuronal loss (or gain) in animal models of these diseases, there are few, if any, readily-available means of rapidly assaying changes in functional synaptic connectivity between neurons in the afflicted brain regions.

The lack of such analytical tools has (1) hindered determination of the location and true extent of disease-related synaptic reorganization as a function of disease progression within interconnected structures of the brain; (2) been an impediment in our ability to fully identify or isolate components of pathogenic circuits within underlying brain regions and decipher mechanisms responsible for rendering these circuits pathogenic; and consequently, (c) retarded the process of planning and designing interventional, preventative, and/or curative strategies for these diseases.

Conventional methods exist to obtain assays of functional synaptic connectivity.

Using these conventional methods, synaptic connectivity is assayed in vitro, usually in acute brain slices, with the aid of the dual- or paired-electrophysiological recording technique. This is effective for assessing gap junctional and synaptic connectivity between pairs of neurons and/or to assess functional properties of the underlying synapses, receptors and connection probabilities.

As depicted in FIG. 1A, post-synaptic currents are evoked in the neuron of interest (generally recorded in the whole-cell voltage-clamp configuration) by depolarizing the presynaptic neuron (recorded in whole-cell current-clamp mode) such that the firing of an action potential evokes a post-synaptic current if the pre- and post-synaptic neurons are connected with each other. The power of the dual-recording technique stems from the fact that it enables studying communication across a single synaptic junction between pairs of interconnected neurons without contamination from extraneous sources (e.g., polysynaptic activity that could arise from stimulating pre-synaptic fibers with an electrode), and, because the presynaptic neuron is under the experimenter's control, its identity and location can be ascertained precisely from electrophysiological data and visualization of a marker with which it can be filled during recording (e.g., biocytin).

However, this technique can be extremely labor-intensive and can severely limit the number of connections that can be tested. Paired-recording can be particularly exasperating when assaying synaptic connectivity in brain regions that are sparsely interconnected and yield a low probability of getting synaptically-coupled pairs of neurons (e.g. neocortex, where the probability of finding a synaptic connection in dual recordings from layer 5 pyramidal neurons can be as low as 10%). Hence, this technique tends to be highly inadequate for measuring the extent and degree of disease-mediated synaptic reorganization following loss of neurons.

Another known method is laser-scanning photostimulation (herein “LSPS”). LSPS is an in vitro technique (originally introduced by Katz and Dalva, 1994) for the focal stimulation of synaptic inputs onto neurons through uncaging of “caged” glutamate in their vicinity. Caged-glutamate is essentially the neurotransmitter glutamate that is rendered inert by a caging molecule. When photolysed, for example by ultra-violet (UV) light, the neurotransmitter becomes liberated or “uncaged”, enabling it to produce its effect, as depicted in FIG. 1B. The amount of glutamate uncaged is proportional to the caged-compound photolysed.

This technique enables “focal stimulation” simply through restriction of the area of photo-stimulation, which is achieved, for example, through the use of a UV laser and by varying its spot diameter. The UV spot can be moved around to uncage glutamate at various locations within the slice (bathed continually with the inert caged-compound) in a random pattern of stimulation that systematically covers the entire region of interest, as depicted in FIG. 2. The major drawbacks of LSPS are that it (1) indiscriminately stimulates all neurons expressing glutamate receptors, and (2) can only probe the small subset of local projections that are preserved in the brain slices.

Accordingly, what is needed is a methodology for assessing diseases and processes that affect the synaptic reorganization of brain circuitry, the methodology used to provide enhanced ability of interventional, preventative or curative strategies thereof. However, in view of the art considered as a whole at the time the present invention was made, it was not obvious to those of ordinary skill how the art could be advanced.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

The long-standing but heretofore unfulfilled need for an improved, rapid and accurate assessment of synaptic activity in the brain circuitry is now met by a new, useful and nonobvious invention.

The current invention provides a platform, or process, for rapid assessment of functional synaptic connectivity in brain circuits based on the Laser Scanning Photo Stimulation (LSPS) technology, which, when implemented successfully, would overcome the barriers presented by the prior art. It is contemplated that the current invention can not only be effective on disease assessment, but also equally amenable to studying normal processes involving synaptic reorganization of brain circuitry such as neural development.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the nature and objects of the invention, reference should be made to the following detailed disclosure, taken in connection with the accompanying drawings, in which:

FIG. 1 depicts prior art schematics of currently available assays of functional synaptic connectivity and the principle underlying LSPS of glutamate, AP action potential and PSC post-synaptic current.

FIG. 1A depicts paired recording technique.

FIG. 1B depicts LSPS;

FIG. 2 depicts a prior art flowchart illustrating the platform for LSPS-assisted evaluation of large-scale disease-related synaptic reorganization according to an embodiment of the current invention;

FIG. 3 depicts an illustration assessing intra-cortical connectivity mediated by the corpus callosum in coronal brain slices using an embodiment of the current invention;

FIG. 4 depicts LSPS of glutamate, an integral component of a platform of the current invention; and

FIG. 5 depicts a scheme for the development of quantifiable measures for disease-related synaptic reorganization based on data acquired by an embodiment of the current invention.

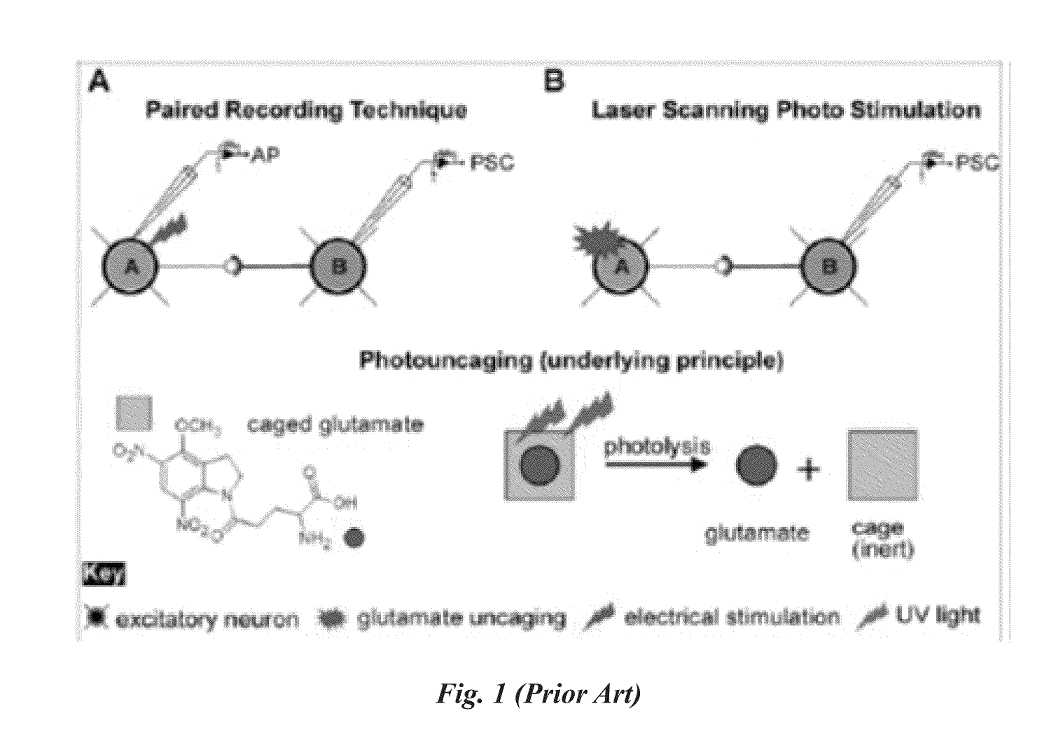

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

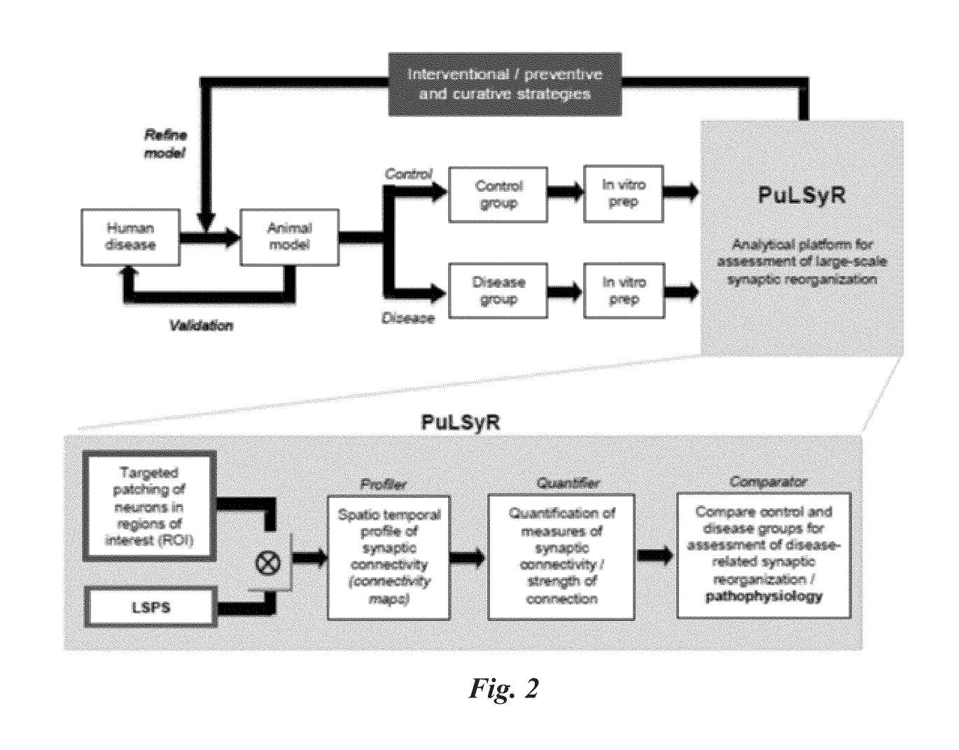

Certain embodiments of the current invention are a photouncaging-assisted evaluation of large-scale synaptic reorganization of brain circuits (herein “PuLSyR”). PuLSyR is the process for LSPS-assisted evaluation of large-scale synaptic reorganization, as depicted in FIG. 2. The term “large-scale” refers to the spatiotemporal scope of the platform that is conducive for assaying connectivity over large areas of interest in the shortest time possible.

PuLSyR involves (1) targeted patching of neurons whose connectivity with others within a predetermined region of interest is in question, and (2) LSPS, a paired-recording technique in which a bolus of neurotransmitter uncaged at the surface of the brain slice diffuses through the tissue to stimulate the presynaptic neuron at its soma (i.e., instead of current applied via a stimulating electrode) causing it to depolarize and fire an action potential. As in paired recordings, the firing of an action potential evokes a post-synaptic current if the pre- and post-synaptic neurons are connected with each other. The neurotransmitter can be uncaged at many locations within the slice simply by moving the UV spot or other photolysing mechanism to those locations for a rapid, near-complete assessment of functional synaptic connectivity of a given species of neurons with neighboring regions of interest. Thus, this method is essentially equivalent to performing multiple paired-recordings simultaneously.

PuLSyR is based on the premise that the degree to which long-range axonal projections between juxtaposed regions of the brain are preserved during the cutting of the slices is what ultimately determines the spatial range of the LSPS assay. Severing long-range axonal connections limits or diminishes the applicability of LSPS for large-scale assessment of synaptic reorganization. Conversely, measures that preserve long-range connectivity between adjacent brain-regions extend or enhance its applicability for this purpose. These measures include (1) quality of the slice preparation, (2) the slicing angle and (3) slice-thickness.

As tissue-handling procedures such as slicing remain invariant between control and disease groups under the PuLSyR protocol, this technique enables direct comparison of synaptic connectivity under the two conditions and can be used for an unbiased assessment of disease-related synaptic reorganization. Preliminary results of the application of PuLSyR for assessment of recurrent circuits in layer II of the medial entorhinal cortex and synaptic reorganization triggered by loss of vulnerable population of neurons in layer 3, in an animal model having temporal lobe epilepsy are very encouraging (Kumar SS et al., J Neurosci 27:1239-1246, 2007, which is hereby incorporated by reference).

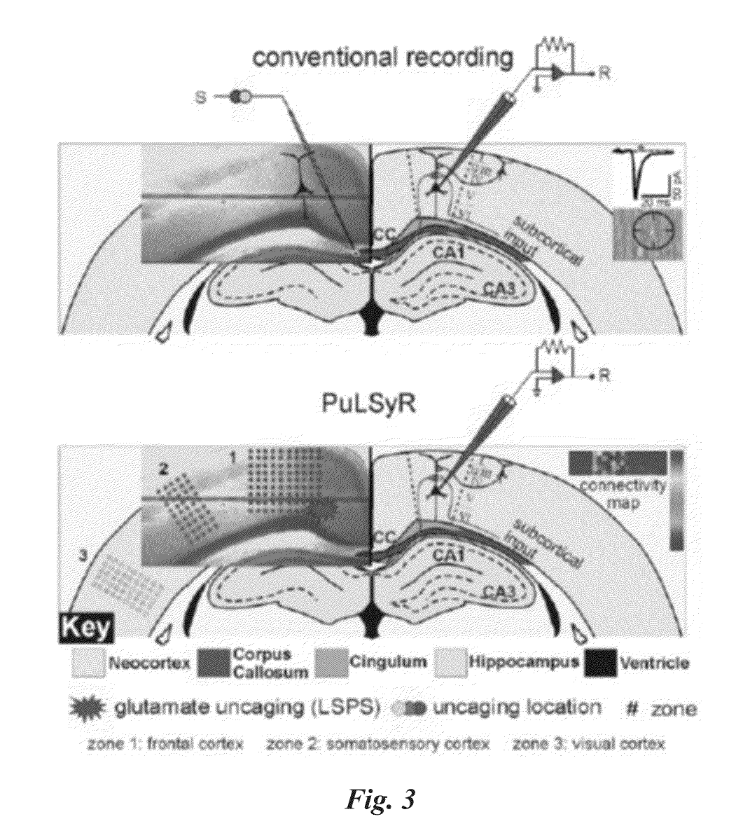

Another example of how this technique can be used for assessing intracortical connectivity mediated by the corpus callosum in coronal brain slices in lieu of the conventional recording method is shown in FIG. 3. FIG. 3 depicts the expansion of the region of interest (scalability) and the rapidity with which synaptic connectivity can be assay when compared to conventional recordings. A similar scheme will also be used to assess intra-hemispheric connectivity and disease-related synaptic reorganization in a mouse model of Alzheimer's disease.

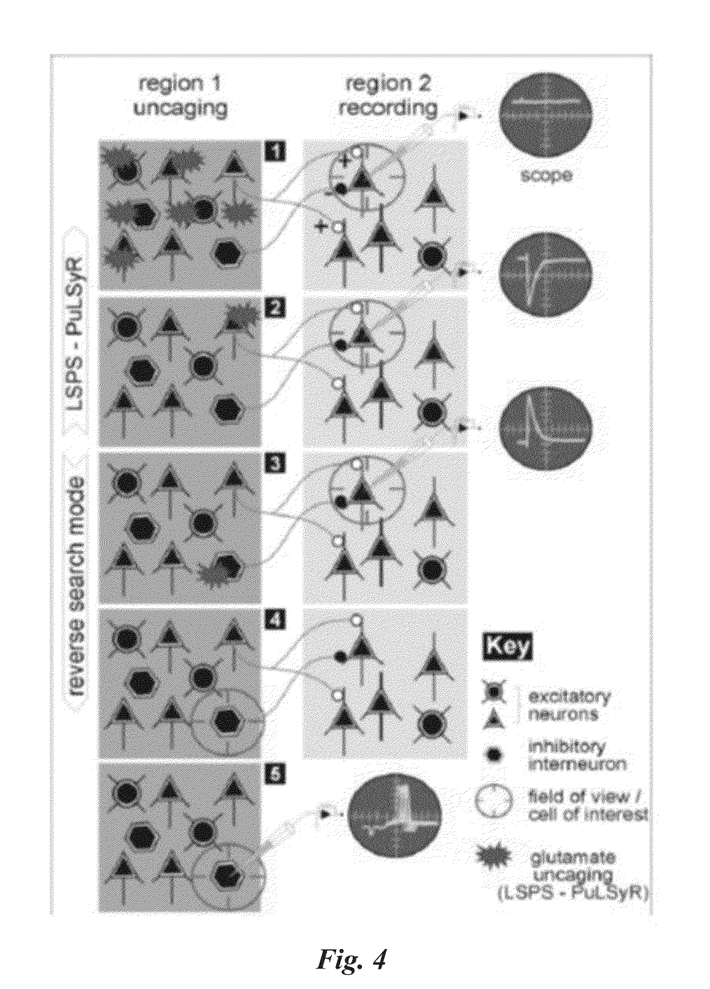

When used in the “reverse search mode”, as depicted in FIG. 4, the LSPS technique offers an additional advantage to the PuLSyR system—one that enables identification of the presynaptic element in a pair of synaptically-connected neurons. “Hot spots” or locations within a slice where glutamate uncaging evokes one or more postsynaptic currents (excitatory postsynaptic currents for excitatory and inhibitory postsynaptic currents for GABAergic neurons) in the recorded neuron are generally where the stomata of the stimulated presynaptic neurons are located.

Knowledge of the location of these hotspots facilitates determination of the whereabouts (i.e., coarse location) of these neurons, especially neuron-types that are sparsely distributed, within a given region of interest. Hence, it is possible to specifically isolate rare GABAergic projection neurons from others in the vicinity in one region, based on recordings of inhibitory postsynaptic currents evoked in pre-identified neurons in another region with which they may be synaptically connected. This is of importance in determining the specific identity of neurons in a region of interest.

Glutamate can be used in reverse-search mode for locating somata of presynaptic neurons among synaptically-connected pairs. Glutamate can be uncaged to focally activate GABAergic neurons while inhibitory synaptic responses are recorded by holding the postsynaptic cell at 0 mV, the reverse potential for EPSCs, as seen in panels 3-5 of FIG. 4.

A number photolabile caged compounds are now available commercially and/or non-commercially and many more can be developed to further enhance the application of the PuLSyr platform. These include caged nucleotides (e.g. ATP, cAMP), proteins (e.g. actin) and neurotransmitters (e.g. glutamate, GABA, Glycine).

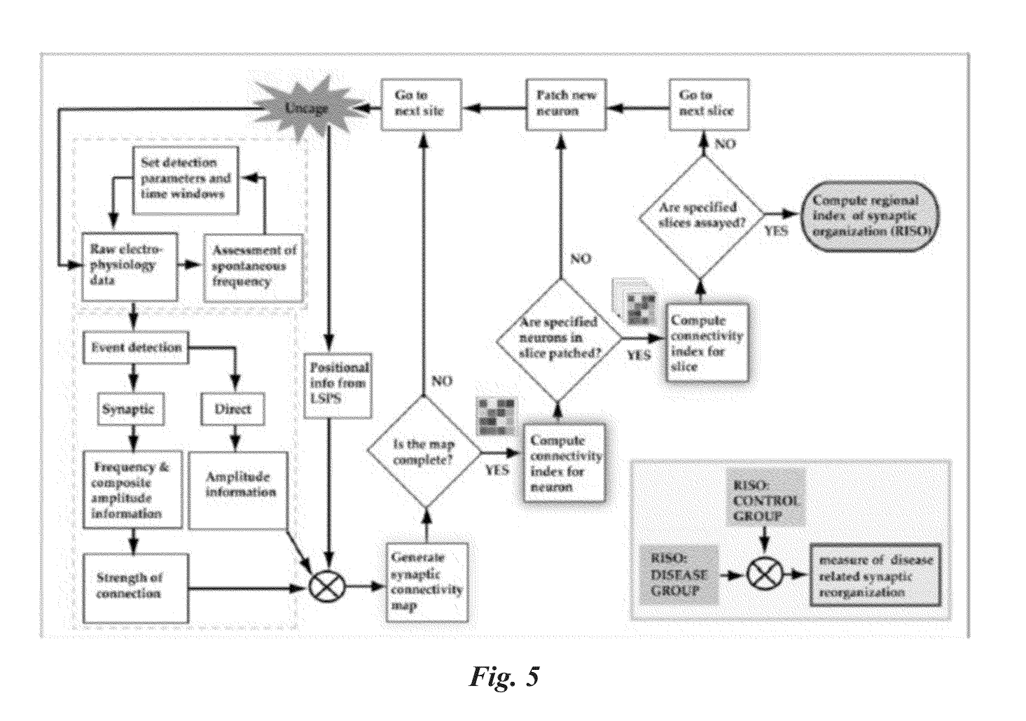

As depicted in FIG. 5, certain embodiments of the current invention include a scheme for the development of 3-dimensional quantifiable measures for disease-related synaptic reorganization based on PuLSyR-acquired data.

It will thus be seen that the objects set forth above, and those made apparent from the foregoing disclosure, are efficiently attained. Since certain changes may be made in the above construction without departing from the scope of the invention, it is intended that all matters contained in the foregoing disclosure or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A method of assessing synaptic reorganization and functional synaptic connectivity in a brain slice with a neurological disorder, comprising the steps of:
patching of targeted neurons, said targeted neurons having connectivity with corresponding neurons in a predetermined region of interest;
uncaging a bolus of neurotransmitters by positioning a photolysing mechanism over said predetermined region of interest in said brain slice;
performing laser scanning photostimulation to detect said bolus of neurotransmitters uncaged at the surface of said brain slice, said bolus of neurotransmitters having diffused through tissue to stimulate presynaptic neurons at a soma of said bolus of neurotransmitters causing said presynaptic neurons to depolarize and fire an action potential, said action potential evoking a post-synaptic current as a result of said bolus of neurotransmitters and said presynaptic neurons being connected to one another.
